(19) United States
(12) Patent Application Publication
(10) Pub. No.: US 2008/0262215 A1
(43) Pub. Date: Oct. 23, 2008

(54) GEMCITABINE PRODUCTION PROCESS

(75) Inventors: Lior ZELIKOVITCH, Mazkeret Batya (IL); Oded FRIEDMAN, Yechiel (IL); Tamir FIZITZKY, Beer Sheva (IL); Josef MANASCU, Omer (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Chemagis Ltd., Bnei Brak (IL)

(21) Appl. No.: 11/738,949

(22) Filed: Apr. 23, 2007

(51) Int. Cl. C07H 19/073

(52) U.S. Cl. 536/28.5

(57) ABSTRACT

Provided is a process for preparing gemcitabine or a salt thereof, which preferably includes selectively precipitating the β-anomer of a 3′,5′-di-O-protected-N⁴-trimethylsilyl-2′-deoxy-2′,2′-difluorocytidine, removing the protecting groups to produce gemcitabine, and, optionally, converting the gemcitabine into a salt. Preferably, the 3′ and 5′ protecting groups are the same or different, and at least one of the 3′ and 5′ protecting groups is cinnamoyl, naphthoyl, naphthylmethylcarbonyl, 2-methylbenzylcarbonyl, 4-methylbenzylcarbonyl or 9-fluorenylmethyloxycarbonyl. Also provided are methods for enriching the β-anomer from an anomeric mixture of a 3′,5′-di-O-protected-N⁴-trimethylsilyl-2′-deoxy-2′,2′-difluorocytidine, e.g., a N⁴-trimethylsilyl-2′-deoxy-2′,2′-difluoro-cytidine-3′,5′-diester, e.g., 3′,5′-dicinnamoyl-N⁴-trimethylsilyl-2′-deoxy-2′,2′-difluorocytidine, using a slurrying process, and methods for converting the β-anomer-enriched product into gemcitabine or a salt thereof.

### BACKGROUND OF THE INVENTION

Gemcitabine HCl, marketed by Eli Lilly under the trademark Gemzar®, is a nucleoside analogue that exhibits antitumor activity and belongs to a general group of chemotherapy drugs known as antimetabolites. Gemcitabine prevents cells from producing DNA and RNA by interfering with the synthesis of nucleic acids, and thus interferes with the growth of cancer cells and slows their growth and spread in the body. Gemcitabine is a synthetic glucoside analog of cytosine, which is chemically described as 4-amino-1-(2-deoxy-2,2-difluoro-β-D-ribofuranosyl)-pyrimidin-2(1H)-one or 2′-deoxy-2′,2′-difluorocytidine (β isomer). Gemcitabine HCl has the following structure:

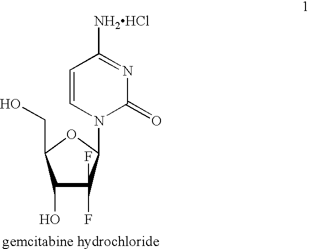

Gemzar® is supplied in vials as the hydrochloride salt in sterile form, for intravenous use, containing either 200 mg or 1 g of gemcitabine HCl (equivalent to the free base) formulated with mannitol (200 mg or 1 g, respectively) and sodium acetate (12.5 mg or 62.5 mg, respectively) as a sterile lyophilized powder. Hydrochloric acid and/or sodium hydroxide may have been added for pH adjustment.

U.S. Pat. No. 4,808,614 (the '614 patent) describes a process for synthetically producing gemcitabine, which process is generally illustrated in Scheme 1.

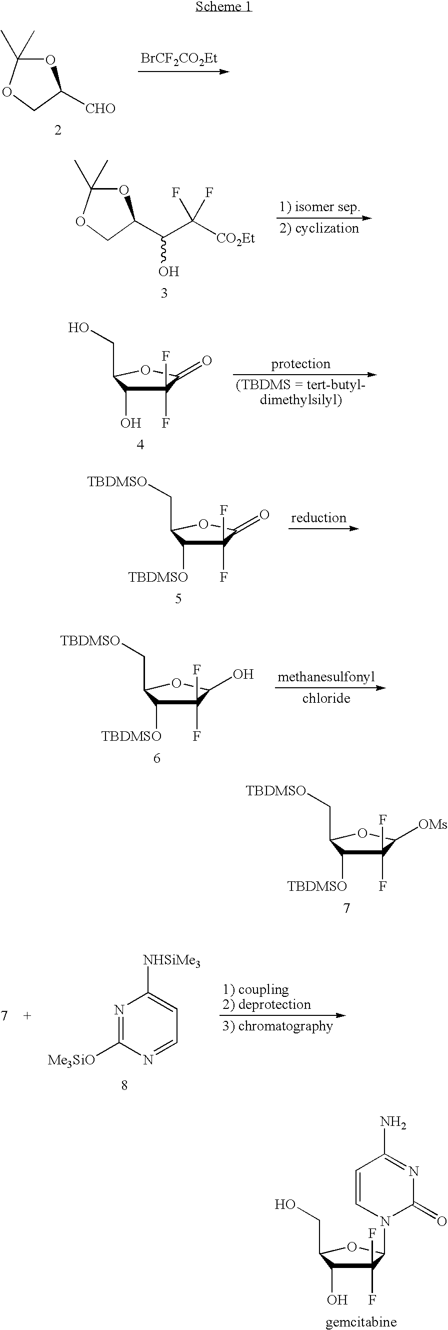

The D-glyceraldehyde ketal 2 is reacted with bromodifluoroacetic acid ethyl ester (BrCF₂COOEt) in the presence of activated zinc, to obtain ethyl 2,2-difluoro-3-hydroxy-3-(2,2-dimethyldioxolan-4-yl)-propionate 3 as a mixture of 3-R and 3-S isomers. The 3-R to 3-S isomer ratio is about 3:1. The 3-R isomer has the stereochemistry required for producing the desired erythro (3-R) ribose structure, and can be separated from the 3-S isomer by chromatography.

The resulting product is cyclized by treatment with an acidic ion exchange resin, such as Dowex 50W-X12, to produce 2-deoxy-2,2-difluoro-D-erythro-pentanoic acid-γ-lactone 4. The hydroxy groups of the lactone are protected with tert-butyldimethylsilyl (TBDMS) protecting groups to obtain the protected lactone 3,5-bis-(tert-butyldimethylsilyloxy)-2-desoxy-2,2-difluoro-1-oxoribose 5, and the product is reduced to obtain 3,5-bis-(tert-butyldimethylsilyl)-2-desoxy-2,2-difluororibose 6.

The 1-position of the carbohydrate is activated by the introduction of a leaving group, e.g., methanesulfonyloxy (mesylate), formed by reacting compound 6 with methanesulfonyl chloride to obtain 3,5-bis-(tert-butyldimethylsilyloxy)-1-methanesulfonyloxy-2-desoxy-2,2-difluororibose 7. The base ring is coupled to the carbohydrate by reacting compound 7 with N,O-bis-(trimethylsilyl)-cytosine 8 in the presence of a reaction initiator, such as trifluoromethanesulfonyloxy trimethylsilane (trimethylsilyl triflate). Removal of the protecting groups and chromatographic purification affords gemcitabine free base.

U.S. Pat. No. 4,526,988 describes a similar process in which the cyclization is carried out by hydrolyzing an alkyl 3-dioxolanyl-2,2-difluoro-3-hydroxy-propionate with a mildly acidic ion exchange resin. See also, Hertel et al. in J. Org. Chem. 53, 2406 (1998).

U.S. Pat. No. 4,965,374 (the '374 patent) describes a process for producing gemcitabine from an intermediate 3,5-dibenzoyl ribo protected lactone of the formula:

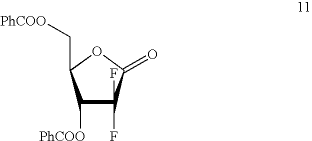

where the desired erythro isomer can be isolated in a crystalline form from a mixture of erythro and threo isomers. The process described in the '374 patent is generally outlined in Scheme 2.

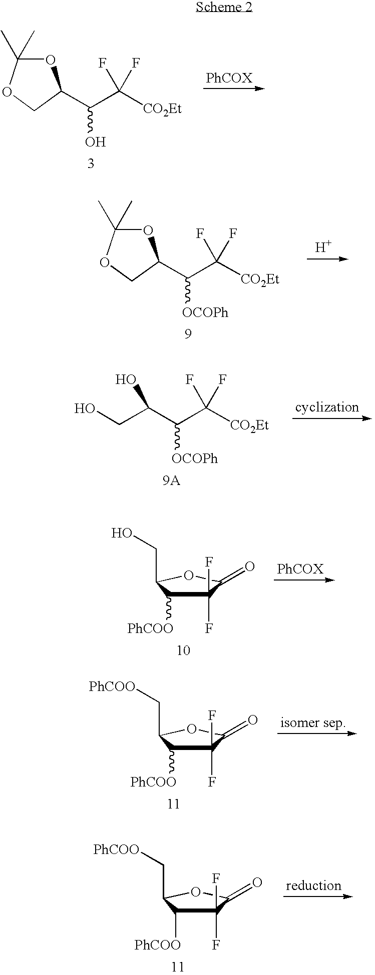

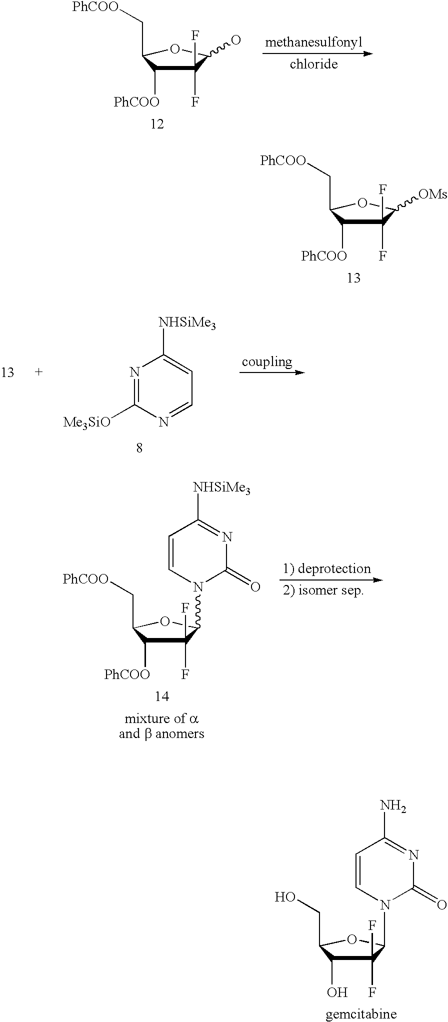

The 3-hydroxy group of compound 3 is esterified with a benzoyl protecting group by reaction with benzoyl chloride, benzoyl bromide, benzoyl cyanide, benzoyl azide, etc. (e.g., PhCOX, wherein X═Cl, Br, CN, or N₃), in presence of a tertiary amine or a catalyst such as 4-dimethylaminopyridine or 4-pyrrolidinopyridine, to obtain ethyl 2,2-difluoro-3-benzoyloxy-3-(2,2-dimethyldioxolan-4-yl)-propionate 9.

The isoalkylidene protecting group of 9 is selectively removed, e.g., by using a strong acid such as concentrated sulfuric acid in ethanol, to produce ethyl-2,2-difluoro-3-benzoyloxy-4,5-dihydroxypentanoate 9A. The product is cyclized to lactone 10 and converted to the dibenzoate ester to produce the lactone 2-deoxy-2,2-difluoropentofuranos-1-ulose-3,5-dibenzoate 11 as a mixture of erythro and threo isomers. The '374 patent describes isolating at least a portion of the erythro isomer from the mixture by selective precipitation. See also, Chou et al., Synthesis, 565-570, (1992).

Compound 11 is then reduced to obtain a mixture of α and β anomers of 2-desoxy-2,2-difluorpentofuranose-dibenzoate 12, which is activated with methane sulfonylchloride to obtain an anomeric mixture of mesylates, 2-deoxy-2,2-difluoro-D-ribofuranosyl-3,5-di-O-benzoyl-1-O-β-methanesulfonate 13, and coupled with N,O-bis(trimethylsilyl)-cytosine 8 to obtain silyl-protected nucleoside 14 as the dibenzoate ester as a mixture of the α- and β-anomers (about a 1:1 α/β anomer ratio). Removal of the esters and silyl protecting group provides a mixture of the β-anomer (gemcitabine) and the α-anomer (about a 1:1 α/β anomer ratio). The '374 patent describes selectively isolating the β-anomer (gemcitabine) by forming a salt of the anomeric mixture, e.g., the hydrochloride or hydrobromide salt, and selectively precipitating to obtain 2′-deoxy-2′,2′-difluorocytidine as the salt in 1:4 α/β ratio. The '374 patent also describes selectively precipitating the β-anomer in free base form in a slightly basic aqueous solution. One such process involves dissolving the 1:1 α/β anomeric mixture in hot acidic water (pH adjusted to 2.5-5.0) and, once the mixture is substantially dissolved, increasing the pH to 7.0-9.0 and allowing the solution to cool, to produce crystals, which are isolated by filtration.

U.S. Pat. No. 5,521,294 (the '294 patent) describes 1-alkylsulfonyl-2,2-difluoro-3-carbamoyl ribose intermediates and intermediate nucleosides derived therefrom. The compounds are reportedly useful in the preparation of 2′-deoxy-2′,2′-difluoro-β-cytidine and other β-anomer nucleosides. The '294 patent teaches, inter alia, that the 3-hydroxy carbamoyl group on the difluororibose intermediate may enhance formation of the desired β-anomer nucleoside derivative. The '294 patent describes converting the lactone 4 to the dibenzoyl mesylate 13, followed by deprotection at the 3 position to obtain the 5-monobenzoyl mesylate intermediate 15, which is reacted with various isocyanates to obtain the compounds of formula 16. The next steps involve coupling and deprotection using methods similar to those described in previous patents. The process and the intermediates 15 and 16 are illustrated by scheme 3 below:

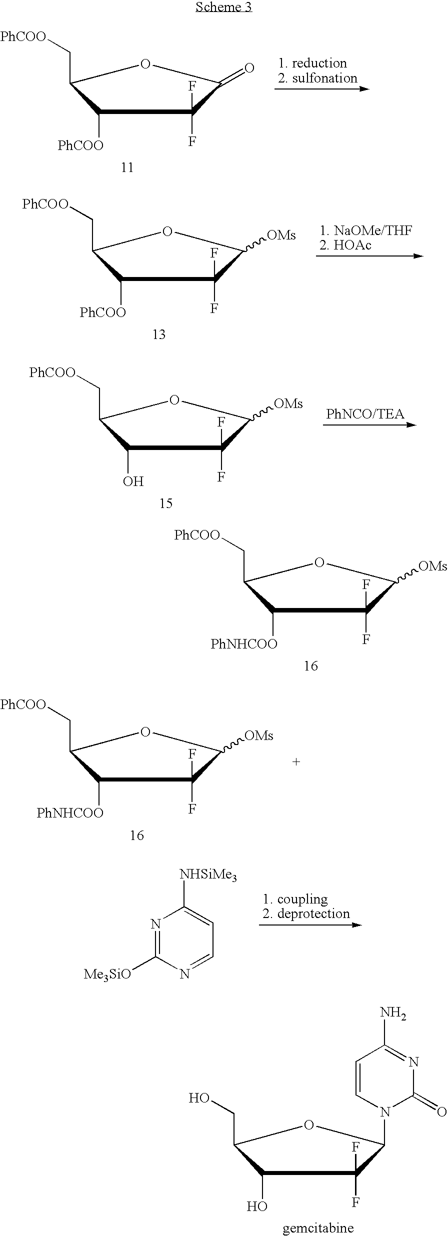

Processes for separating anomeric mixtures of alkylsulfonate intermediates also have been described. U.S. Pat. Nos. 5,256,797 and 4,526,988 describe processes for separating anomers of 2-deoxy-2,2-difluoro-D-ribofuranosyl-1-alkylsulfonates, and U.S. Pat. No. 5,256,798 describes a process for obtaining α-anomer-enriched ribofuranosyl sulfonates.

Other intermediates that may be useful for preparing gemcitabine have been disclosed. For instance, U.S. Pat. No. 5,480,992 describes anomeric mixtures of 2,2-difluororibosyl azide and corresponding amine intermediates that can be prepared, e.g., by reacting a 2-deoxy-2,2-difluoro-D-ribofuranosyl-3,5-di-O-benzoyl-1-O-β-methanesulfonate with an azide nucleophile, such as lithium azide, to obtain the azide. Reduction of the azide produces the corresponding amine, which can be synthetically converted into a nucleoside. See also U.S. Pat. Nos. 5,541,345 and 5,594,155.

Other known intermediates include, e.g., tritylated intermediates (U.S. Pat. No. 5,559,222), 2-deoxy 2,2-difluoro-β-D-ribo-pentopyranose (U.S. Pat. No. 5,602,262), 2-substituted-3,3-difluorofuran intermediates (U.S. Pat. No. 5,633,367), and α,α-difluoro-β-hydroxy thiol esters (U.S. Pat. Nos. 5,756,775 and 5,912,366).

WO 2007/027564 (hereinafter the '564 application) describes a process for preparing gemcitabine or a salt thereof, which includes separating a N⁴-protected-2′-deoxy-2′,2′-difluoro-cytidine-3′,5′-diester from an anomeric mixture thereof; removing the 3′-ester, the 5′-ester and the N-protecting group; and optionally forming a salt. The 3′-ester and 5′-ester can include cinnamoyl, naphthoyl, naphthylmethylcarbonyl, 2-methylbenzylcarbonyl, 4-methylbenzylcarbonyl and 9-fluorenylmethyloxycarbonyl esters. The '564 application also describes 2-deoxy-2,2-difluoro-D-erythro-pentofuranos-1-ulose-3,5-diester intermediates and methods for producing such intermediates.

There are inherent problems associated with the production of gemcitabine, particularly for processes that require the production and separation of isomers, which tend to be problematic on a commercial scale. Accordingly, there is a need for improved methods of preparing gemcitabine and intermediates thereof, which facilitate the production of gemcitabine, particularly on a commercial scale. The present invention provides such methods.

### BRIEF SUMMARY OF THE INVENTION

The present invention provides a process for preparing gemcitabine or a salt thereof, which preferably includes selectively precipitating the β-anomer from an anomeric mixture of a 2′-deoxy-2′,2′-difluoro-3′,5′-di-O-protected-cytidine, removing the protecting groups to produce gemcitabine and, optionally, converting the gemcitabine to a salt. The N⁴-protected-2′-deoxy-2′,2′-difluoro-3′,5′-di-O-protected-cytidine used in the process of the present invention as starting material, is preferably a N⁴-trimethylsilyl-2′-deoxy-2′,2′-difluoro-3′,5′-di-O-protected-cytidine.

Exemplary N⁴-protected-2′-deoxy-2′,2′-difluoro-3′,5′-di-O-protected-cytidines that can be used as gemcitabine as precursors in accordance with the present invention include compounds of the formula 17 (Scheme 4), wherein R and R′ are the same or different and at least one of R or R′ is phenyl, 2-phenylethenyl (thus forming a cinnamoyl ester), 1-naphthyl, 1-naphthylmethyl, 2-methylbenzyl, 2-methylbenzyl or 4-methylbenzyl. An exemplary process of the present invention includes selectively precipitating the β-anomer from an anomeric mixture of 2′-deoxy-2′,2′-difluoro-3′,5′-dicinnamoyl-cytidine, removing the 3′ and 5′ protecting groups to produce gemcitabine and, optionally, converting the gemcitabine into a salt (e.g., gemcitabine hydrochloride).

The N⁴-protected-2′-deoxy-2′,2′-difluoro-3′,5′-di-O-protected-cytidine precursors can be synthesized by any suitable method. In one embodiment, the present invention provides a process for producing gemcitabine from lactols of formulae 12A, which process preferably includes:

a) reacting lactol 12A, with p-toluenesulfonyl chloride (tosyl chloride) in the presence of a base to obtain a tosylate intermediate of formula 13A;

b) coupling the compound of formula 13A with N,O-bis-(trimethylsilyl)-cytosine in an organic solvent, optionally in the presence of a catalyst, to obtain a mixture of α and β anomers of the 3′,5′-diprotected-N⁴-trimethylsilyl-2′-deoxy-2′,2′-difluorocytidine 17;

c) removing the trimethylsilyl group and selectively precipitating the β-anomer of the 2′-deoxy-2′,2′-difluoro-3′,5′-di-O-protected-cytidine, thus allowing for separation of the two isomers, e.g., by filtration;

d) removing the ester protecting groups, e.g., by hydrolysis, to obtain gemcitabine;

e) optionally, converting the gemcitabine to a salt thereof; and

f) optionally further purifying the gemcitabine salt, e.g., by crystallization.

Thus, the present invention provides a process for obtaining gemcitabine or a salt thereof in high purity and yield from a 2-desoxy-2,2-difluoropentofuranose-diester (e.g., 2-desoxy-2,2-difluoropentofuranose-dicinnamate) having the general formulae 12A. The compound 2-desoxy-2,2-difluoropentofuranose-dicinnamate is a particularly useful intermediate for synthesizing and obtaining highly pure β-isomer of 2′-deoxy-2′,2′-difluoro-3′,5′-dicinnamoyl-cytidine using common organic solvents (e.g., ethyl acetate) in accordance with the present invention.

In one embodiment, the coupling reaction is carried out in a solvent such as, e.g., 1,2-dichloroethane. Optionally, the coupling reaction can be facilitated by carrying out the reaction in the presence of a suitable catalytic reagent such as, for example, trimethylsilyl triflate (Me₃SiOTf).

Removal of the protecting groups can be carried out using any suitable conditions, which can include, for example, hydrolytic conditions, e.g., basic hydrolysis, e.g., aqueous solution of sodium bicarbonate (NaHCO₃) for removing the trimethylsilyl group and about 16% ammonia in methanol for removing the ester groups.

In one embodiment of the present invention, precipitation of crude 2′-deoxy-2′,2′-difluoro-3′,5′-dicinnamoyl-cytidine from ethyl acetate after the N⁴-trimethylsilyl protecting group had been removed by treatment with aqueous solution of sodium bicarbonate, directly affords predominantly the β-anomer of 2′-deoxy-2′,2′-difluoro-3′,5′-dicinnamoyl-cytidine in about a 73:12 mixture of the β:α-anomeric mixture of 2′-deoxy-2′,2′-difluoro-3′,5′-dicinnamoyl-cytidine. Precipitating crude 2′-deoxy-2′,2′-difluoro-3′,5′-dicinnamoyl-cytidine from a smaller volume of ethyl acetate relative to the quantity of the starting material 2-deoxy-2,2-difluoro-D-ribofuranose-3,5-dicinnamate-1-p-toluenesulfonate, after the N⁴-trimethylsilyl protecting group had been removed by treatment with concentrated aqueous solution of sodium bicarbonate, can produce a α:β-anomeric mixture of 2′-deoxy-2′,2′-difluoro-3′,5′-dicinnamoyl-cytidine in a high yield, e.g., 99.9%. Thus, the present invention provides a process for separating the β-anomer of the 3,5-diprotected-2′-deoxy-2′,2′-difluorocytidine by selective precipitation. Preferably, the selective precipitation process comprises:

a) dissolving a crude mixture of 3,5-diprotected-N⁴-trimethylsilyl-2′-deoxy-2′,2′-difluorocytidine in an organic solvent and extracting with water;

b) adding an aqueous solution containing a base to the organic phase to produce a precipitate, optionally with mixing;

c) collecting the precipitate, e.g., by filtration; and

d) optionally washing the precipitate, e.g., with an organic solvent and drying, e.g., at elevated temperature.

The present invention further provides a process for enriching the content of the β-anomer of a 3′,5′-diprotected-2′-deoxy-2′,2′-difluorocytidine, which process preferably includes:

a) slurrying the 3,5-diprotected-2′-deoxy-2′,2′-difluorocytidine in an organic solvent;

b) collecting the solid, e.g., by filtration;

c) optionally washing the solid, e.g., with an organic solvent; and

d) optionally drying, e.g., at elevated temperature.

In accordance with the present invention, the 3′,5′-diprotected-2′-deoxy-2′,2′-difluorocytidine (e.g., 2′-deoxy-2′,2′-difluoro-3′,5′-dicinnamoyl-cytidine) can be obtained in high yield, e.g., at least about 98% yield. In accordance with the present invention, gemcitabine or a salt thereof is obtained in a purity of at least about 99%, preferably in a purity of at least about 99.5% and more preferably in a purity of at least about 99.9%.

### DETAILED DESCRIPTION OF THE INVENTION

The present invention in predicated, at least in part, on the surprising discovery that it is possible to obtain the β-anomer (e.g., a product that is enriched in β-anomer) of 2′-deoxy-2′,2′-difluoro-3′,5′-di-O-protected-cytidine precursors by “reverse” precipitation, i.e., by selectively precipitating the β-anomer from an anomeric mixture. The selective precipitation process of the preset invention can be achieved by controlling the solvent and solvent volume in the purification process. As demonstrated in the '564 application, a crude 3′,5′-diprotected-N⁴-trimethylsilylacetyl-2′-deoxy-2′,2′-difluorocytidine, e.g., N⁴-trimethylsilylacetyl-2′-deoxy-2′,2′-difluoro-3′,5′-dicinnamoyl-cytidine can be crystallized from a mixture of dichloroethane and methanol to obtain the 60 -anomer of N⁴-trimethylsilylacetyl-2′-deoxy-2′,2′-difluoro-3′,5′-dicinnamoyl-cytidine, and by concentrating the remaining liquor to dryness, the crude β-anomer of N⁴-trimethylsilylacetyl-2′-deoxy-2′,2′-difluoro-3′,5′-dicinnamoyl-cytidine is obtained.

However, the applicants have found that by precipitating crude 3′,5′-di-O-protected-2′-deoxy-2′,2′-difluorocytidine, e.g., 2′-deoxy-2′,2′-difluoro-3′,5′-dicinnamoyl-cytidine, from ethyl acetate, after the N⁴-trimethylsilyl group had been removed by treatment with aqueous solution of sodium bicarbonate, the crude β-anomer of 2′-deoxy-2′,2′-difluoro-3′,5′-dicinnamoyl-cytidine can be directly obtained as a precipitate containing about a 73:12 mixture of the β:α-anomeric mixture of 2′-deoxy-2′,2′-difluoro-3′,5′-dicinnamoyl-cytidine (see example 2). It has also been found that precipitating crude 2′-deoxy-2′,2′-difluoro-3′,5′-dicinnamoyl-cytidine from a smaller volume of ethyl acetate relative to the quantity of the starting material 2-deoxy-2,2-difluoro-D-ribofuranose-3,5-dicinnamate-1-p-toluenesulfonate, after the N⁴-trimethylsilyl group had been removed by treatment with concentrated aqueous solution of sodium bicarbonate, can afford an α:β-anomeric mixture (about 43:52 α:β) of 2′-deoxy-2′,2′-difluoro-3′,5′-dicinnamoyl-cytidine in high yield (see example 3), which can be used as a convenient precursor for obtaining the β-anomer, e.g., by selective (“reverse”) precipitation in accordance present invention.

In a preferred embodiment, the present invention provides a process for preparing gemcitabine or a salt thereof, which preferably includes selectively precipitating the β-anomer of 2′-deoxy-2′,2′-difluoro-3′,5′-dicinnamoyl-cytidine of formula 18, wherein R and R′ are the same or different and at least one of R and R′ is phenyl, 2-phenylethenyl (thus forming a cinnamoyl ester), 1-naphthyl, 1-naphthylmethyl, 2-methylbenzyl, 2-methylbenzyl or 4-methylbenzyl, removing the protecting groups to produce gemcitabine and, optionally, converting the gemcitabine to a salt. The process of the present invention can be conveniently utilized for obtaining highly pure gemcitabine, and the precursors employed in accordance with the present invention can be easily synthesized. An exemplary process of preparing gemcitabine in accordance with the present invention is detailed in Scheme 4 below.

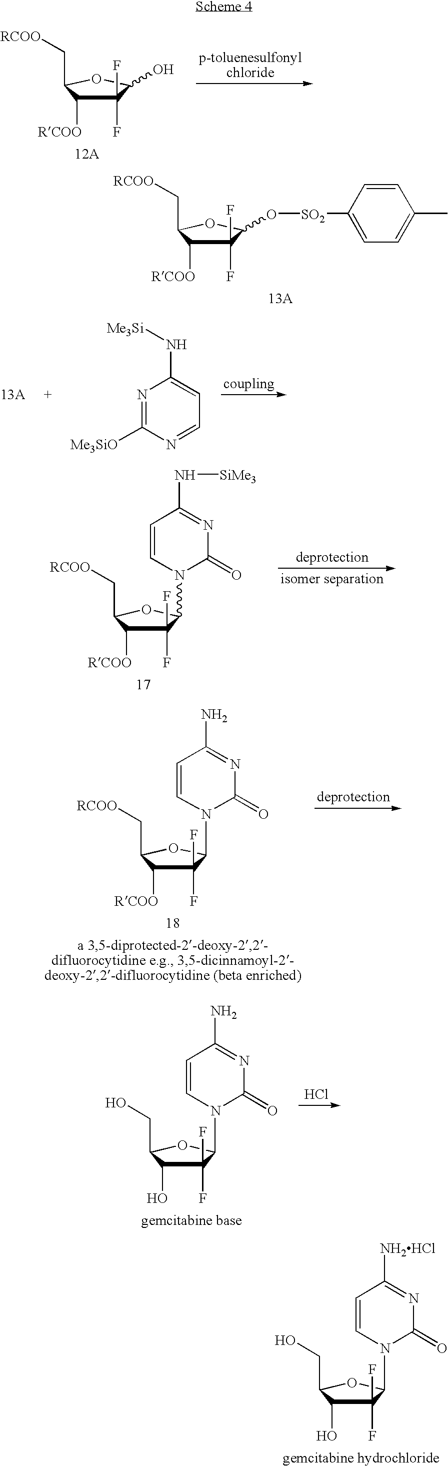

Thus, gemcitabine can be prepared from lactols of the formulae 12A by a process that includes:

a) reacting the lactol intermediate 12A with p-toluenesulfonyl chloride (tosyl chloride) in the presence of a base to obtain the sulfonate intermediate of the formula 13A;

b) coupling the compound of the formula 13A with N,O-bis-(trimethylsilyl)cytosine, preferably at ambient temperature using a catalyst in an organic solvent, to obtain a mixture of α and β anomers of the 3′,5′-diprotected-N⁴-trimethylsilyl-2′-deoxy-2′,2′-difluorocytidine 17;

c) removing the trimethylsilyl group and selectively precipitating the β-anomer of the 3′,5′-diprotected-2′-deoxy-2′,2′-difluorocytidine and isolating the β-anomer, e.g., by filtration;

d) removing the protecting groups, e.g., by hydrolysis, to obtain gemcitabine;

e) optionally converting the gemcitabine into a salt thereof; and

f) optionally purifying the gemcitabine salt, e.g., by crystallization.

Thus, the present invention provides a process for conveniently obtaining gemcitabine or a salt thereof in high purity and yield from 2-desoxy-2,2-difluoropentofuranose-diester (e.g., 2-desoxy-2,2-difluoropentofuranose-dicinnamate) having the general formulae 12A. The compound 2-desoxy-2,2-difluoropentofuranose-dicinnamate is particularly useful intermediate for obtaining high yields of 2′-deoxy-2′,2′-difluoro-3′,5′-dicinnamoyl-cytidine from which highly pure β-isomer can be obtained using commonly-used organic solvents (e.g., ethyl acetate) in accordance with the present invention.

The present invention further provides the novel sulfonate intermediates of the formula 13A

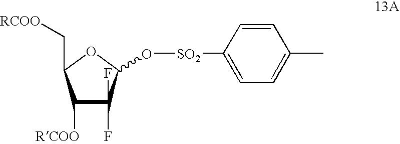

wherein R and R′ are the same or different and at least one of R and R′ is phenyl, 2-phenylethenyl (thus forming a cinnamoyl ester), 1-naphthyl, 1-naphthylmethyl, 2-methylbenzyl, 2-methylbenzyl or 4-methylbenzyl, e.g., 2-deoxy-2,2-difluoro-D-ribofuranose-3,5-dicinnamate-1-p-toluenesulfonate.

The coupling reaction, e.g., as depicted in Scheme 4, can be carried out in any suitable solvent, which can include, for example, one or more organic solvents selected from acetonitrile, ethyl acetate, n-butyl acetate, chloroform, 1,2-dichloro-ethane, toluene, xylenes, and the like, and mixtures thereof. In one embodiment, the coupling reaction is carried out in 1,2-dichloroethane. Optionally, the coupling reaction can be facilitated by using a suitable catalytic reagent such as, for example, trimethylsilyl triflate (Me₃SiOTf).

Removal of the protecting groups can be carried out by using any suitable conditions, which can include, for example, hydrolytic conditions, e.g., basic hydrolysis, e.g., aqueous solution of sodium bicarbonate (NaHCO₃) for removing the trimethylsilyl group and about 16% ammonia in methanol for removing the ester groups.

Precipitation of crude 2′-deoxy-2′,2′-difluoro-3′,5′-dicinnamoyl-cytidine from ethyl acetate directly affords predominantly the β-anomer of 2′-deoxy-2′,2′-difluoro-3′,5′-dicinnamoyl-cytidine in about 73:12 β:α-anomeric mixture. On the other hand, precipitating the crude 2′-deoxy-2′,2′-difluoro-3′,5′-dicinnamoyl-cytidine from a smaller volume of ethyl acetate relative to the quantity of the starting material 2-deoxy-2,2-difluoro-D-ribofuranose-3,5-dicinnamate-1-p-toluenesulfonate conveniently affords a α:β-anomeric mixture of 2′-deoxy-2′,2′-difluoro-3′,5′-dicinnamoyl-cytidine in high yield, e.g., 99.9% yield, which can be used as a precursor for obtaining the β-anomer. Thus, the present invention provides a process for separating the β-anomer from an anomeric mixture of a 3′,5′-diprotected-2′-deoxy-2′,2′-difluorocytidine by a selective precipitation process, which preferably includes:

a) dissolving the crude mixture of 3′,5′-diprotected-N⁴-trimethylsilyl-2′-deoxy-2′,2′-difluorocytidine in an organic solvent and extracting with water;

b) adding an aqueous solution containing a base to the organic phase to produce a precipitate, optionally with mixing;

c) collecting the precipitate, e.g., by filtration; and

d) optionally washing the precipitate, e.g., with an organic solvent and drying, e.g., at elevated temperature.

Suitable organic solvents that can be used for precipitating the 3′,5′-di-O-protected-2′-deoxy-2′,2′-difluorocytidine include, for example, dichloromethane, chloroform, ethyl acetate, 1-propyl acetate, 2-propyl acetate, butyl acetate, tert-butyl acetate, o-xylene, m-xylene, o-dichlorobenzene, toluene, and the like, and mixtures thereof. A preferred solvent for precipitating the β-anomer of the 3′,5′-diprotected-2′-deoxy-2′,2′-difluorocytidine is ethyl acetate.

Suitable bases, which can be used in the process of the present invention, include, for example, sodium carbonate, sodium bicarbonate, potassium carbonate, potassium bicarbonate, and the like. A preferred base is sodium bicarbonate.

In another embodiment, the present invention provides a process for enriching the content of the β-anomer from an anomeric mixture of a 3′,5′-di-O-protected-2′-deoxy-2′,2′-difluorocytidine (e.g., from an anomeric mixture of a 3′,5′-di-cinnamoyl-2′-deoxy-2′,2′-difluorocytidine), which process preferably includes:

a) slurrying the 3′,5′-di-O-protected-2′-deoxy-2′,2′-difluorocytidine (e.g., a 3′,5′-di-cynnamoyl-protected-2′-deoxy-2′,2′-difluorocytidine) in an organic solvent;

b) collecting the solid, e.g., by filtration;

c) optionally washing the solid, e.g., with an organic solvent; and

d) optionally drying, e.g., at elevated temperature.

Suitable solvents that can be used for slurrying the anomeric mixture of the 2′-deoxy-2′,2′-difluoro-3′,5′-di-O-protected-cytidine (e.g., 3′,5′-cinnamoyl-2′-deoxy-2′,2′-difluorocytidine), in order to increase the content of the β-anomer, include dichloromethane, ethyl acetate, methanol, 2-propanol, acetone, acetonitrile, and the like, and mixtures thereof.

The ratio between the 2′-deoxy-2′,2′-difluoro-3′,5′-di-O-protected-cytidine (e.g., 3′,5′-dicinnamoyl-2′-deoxy-2′,2′-difluorocytidine) and the solvent in the slurrying process preferably is at least about 1:1 (g/ml), and more preferably is at least about 1:10 (g/ml).

In accordance with the processes of the present invention (e.g., for selective precipitation the β-anomer as described herein), exemplary 3′,5′-di-O-protected-2′-deoxy-2′,2′-difluorocytidine precursors can include, e.g., 3′,5′-dicinnamoyl-2′-deoxy-2′,2′-difluorocytidine, 3′,5′-dinaphthoyl-2′-deoxy-2′,2′-difluorocytidine, 3′,5′-dinaphthylmethyl-2′-deoxy-2′,2′-difluorocytidine, 3′,5′-di-2-methylbenzyl-2′-deoxy-2′,2′-difluorocytidine, and 3′,5′-di-4-methylbenzyl-2′-deoxy-2′,2′-difluorocytidine. A preferred 3′,5′-di-O-protected-2′-deoxy-2′,2′-difluorocytidine precursor is 3′,5′-dicinnamoyl-2′-deoxy-2′,2′-difluorocytidine.

In accordance with the processes of the present invention (e.g., for selective enriching the β-anomer as described herein), exemplary 3′,5′-di-O-protected-2′-deoxy-2′,2′-difluorocytidine precursors can include, e.g., 3′,5′-dicinnamoyl-2′-deoxy-2′,2′-difluorocytidine, 3′,5′-dinaphthoyl-2′-deoxy-2′,2′-difluorocytidine, 3′,5′-dinaphthylmethyl-2′-deoxy-2′,2′-difluorocytidine, 3′,5′-di-2-methylbenzyl-2′-deoxy-2′,2′-difluorocytidine, and 3′,5′-di-4-methylbenzyl-2′-deoxy-2′,2′-difluorocytidine. A preferred 3′,5′-di-O-protected-2′-deoxy-2′,2′-difluorocytidine precursor is 3′,5′-dicinnamoyl-2′-deoxy-2′,2′-difluorocytidine.

In accordance with the present invention, the 3,5-diprotected-2′-deoxy-2′,2′-difluorocytidine e.g., 2′-deoxy-2′,2′-difluoro-3′,5′-dicinnamoyl-cytidine, can be obtained in high yield, e.g., at least about 98% yield. In accordance with the present invention, gemcitabine or a salt thereof is obtained in a purity of at least about 99%, preferably in a purity of at least about 99.5% and more preferably in a purity of at least about 99.9%.

### EXAMPLES

Although, the following examples illustrate the practice of the present invention in some of its embodiments, the examples should not be construed as limiting the scope of the invention. Other embodiments will be apparent to one skilled in the art from consideration of the specification and examples.

### Example 1

This example demonstrates the preparation of 2-deoxy-2,2-difluoro-D-ribofuranose-3,5-dicinnamate-1-p-toluenesulfonate.

Crude 2-deoxy-2,2-difluoro-D-riboufuranose-3,5-dicinnamate (2.5 g, 6 mmol) was dissolved in dichloromethane (20 ml) in a round flask, and diethylamine (0.7 g, 9.6 mmol) was added followed by p-toluenesulfonyl chloride (1.32 g, 6.92 mmol), which was added drop wise while cooling to 0-5° C. The mixture was stirred for 1 hour, and washed with 1N HCl (15 ml), concentrated solution of NaHCO₃ (15 ml), and dried over MgSO₄. The solvent was distilled off under reduced pressure to obtain crude 2-deoxy-2,2-difluoro-D-ribofuranose-3,5-dicinnamate-1-p-toluenesulfonate as light oil. Yield: 3.22 g, (5.6 mmol), 93%.

### Example 2

This example demonstrates the preparation of 3′,5′-dicinnamoyl-2′-deoxy-2′,2′-difluorocytidine.

Dry 1,2-dichloroethane (800 ml) was added to N,O-bis(trimethylsilyl)-cytosine (136 g, 487 mmol) under nitrogen blanket to produce a clear solution, followed by adding trimethylsilyl triflate (Me₃SiOTf), (100 ml, 122.8 g, 520 mmol) and stirred for 30 minutes. A solution of 2-deoxy-2,2-difluoro-D-ribofuranose-3,5-dicinnamate-1-p-toluenesulfonate (128 g, 224 mmol) in 1,2-dichloroethane (400 ml) was added drop wise, and the mixture was refluxed overnight. After cooling, the solvent was distilled off to obtain crude 3,5-dicinnamoyl-N⁴-trimethylsilyl-2′-deoxy-2′,2′-difluorocytidine as a light yellow solid. The residue was dissolved in ethyl acetate (1600 ml) and washed 3 times with water (3×400 ml). The ethyl acetate phase was mixed with concentrated solution of NaHCO₃ (800 ml) for about 5 minutes, and then the mixture was set aside for about 20 minutes without stirring. The thus formed solid, which was precipitated in the inter-phase of the two layers, was filtered off and washed with 60 ml of ethyl acetate. The solid was dried under reduced pressure to obtain 116.7 g (223 mmol, 99.5%) of the crude 3′,5′-dicinnamoyl-2′-deoxy-2′,2′-difluorocytidine containing 73.3% of the β-anomer and 11.8% of the α-anomer.

### Example 3

This example demonstrates the preparation of 3′,5′-dicinnamoyl-2′-deoxy-2′,2′-difluorocytidine.

Dry 1,2-dichloroethane (1.5 L) was added to bis(trimethylsilyl)cytosine (417 g, 1.49 mol) under nitrogen blanket to produce a clear solution followed by adding trimethylsilyl triflate (Me₃SiOTf), (300 ml, 368.4 g, 1.56 mol) and stirred for 30 minutes. A solution of 2-deoxy-2,2-difluoro-D-ribofuranose-3,5-dicinnamate-1-p-toluenesulfonate (384 g, 673 mmol) in 1,2-dichloroethane (1.2 L) was added drop wise, and the mixture was refluxed overnight. After cooling, the solvent was distilled off to obtain crude 3,5-dicinnamoyl-N⁴-trimethylsilyl-2′-deoxy-2′,2′-difluorocytidine as a light yellow solid. The residue was dissolved in ethyl acetate (2.4 L) and washed 3 times with water (3×1.2 L). The ethyl acetate phase was mixed with concentrated solution of NaHCO₃ (1.34 L) for about 20 minutes. The thus formed solid, which was precipitated in the inter-phase of the two layers, was filtered off and washed with 180 ml of ethyl acetate. The solid was dried under reduced pressure to obtain 346.5 g (0.66 mol, 99.9% yield) of the crude 3′,5′-dicinnamoyl-2′-deoxy-2′,2′-difluorocytidine containing 43% of the β-anomer and 52% of the α-anomer.

### Example 4

This example demonstrates the preparation of gemcitabine hydrochloride.

To a solution of ammonia-methanol (15.8%, 4.57 L), the crude 3,5-dicinnamoyl-2′-deoxy-2′,2′-difluorocytidine of example 3 was added (346.5 g, 0.66 mol), and stirred at ambient temperature for 6 hours. The mixture was concentrated to afford a light yellow solid (306 g). Purified water (3 L) was added to the solid, followed by addition of ethyl acetate (1.8 L), and stirring was maintained for about 10 minutes. The aqueous layer was separated and the organic layer was extracted with water (1.05 L). The aqueous layers were combined and water was removed by evaporation under reduced pressure to obtain an oil (154.7 g). Water was added (660 ml) and the mixture was heated to 50-55° C. to dissolve the solid. The mixture was cooled to 5-10° C. during about one hour and mixed for about 16 hours at that temperature. The thus formed solid was filtered and dried to afford 46.75 g (0.177 mol), containing 98% of the β-anomer and 1.3% of the α-anomer. 0.5N HCl (936 ml) was added followed by addition of dichloromethane (300 ml) with stirring. The water phase was separated and the aqueous phase was washed with dichloromethane (300 ml). After filtration, the aqueous phase was concentrated to dryness under reduced pressure to obtain gemcitabine hydrochloride as a solid (46.9 g). The solid was dissolved in water (187 ml) at ambient temperature and the mixture was heated to 50° C. to afford a clear solution and cooled to ambient temperature. Acetone (1.4 L) was added and stirring was maintained for about one hour. Then, the precipitate was collected by filtration and washed twice with acetone (2×30 ml) and dried at 45° C. under vacuum to obtain 39.2 g of gemcitabine hydrochloride, containing 99.9% of the β-anomer

### Example 5

This example demonstrates the preparation of gemcitabine hydrochloride.

To a solution of ammonia-methanol (about 15.8%, 1.35 L), the crude 3′,5′-dicinnamoyl-2′-deoxy-2′,2′-difluorocytidine prepared as described in example 2 was added (96 g, 183.4 mmol), and stirred at ambient temperature for 4 hours. The mixture was concentrated to afford a light yellow solid (80.5 g). Purified water (1 L) was added to the solid, followed by addition of ethyl acetate (600 ml), and stirring was maintained for about 10 minutes. The aqueous layer was separated and the organic layer was extracted with water (350 ml). The aqueous layers were combined and water was removed by evaporation under reduced pressure to obtain an oil (46.4 g). Water was added (220 ml) and the mixture was heated to 50-55° C. to dissolve the solid. The mixture was cooled to 0-5° C. during about one hour and mixed for about 16 hours at that temperature. The thus formed solid was filtered and dried to afford 11.1 g of gemcitabine free base. 0.5N HCl (240 ml) was added followed by addition of dichloromethane (100 ml) with stirring. The water phase was separated and the aqueous phase was washed with dichloromethane (300 ml). After filtration, the aqueous phase was concentrated to dryness under reduced pressure to obtain gemcitabine hydrochloride as a solid (12.0 g). The solid was dissolved in water (48 ml) at ambient temperature and the mixture was heated to 50° C. to afford a clear solution and cooled to ambient temperature. Acetone (360 ml) was added and stirring was maintained for about one hour. Then, the precipitate was collected by filtration and washed twice with acetone (2×30 ml) and dried at 45° C. under vacuum to obtain 9.9 g of gemcitabine hydrochloride, containing 99.6% of the β-anomer.

### Example 6

This example demonstrates the slurrying procedure of the 3′,5′-dicinnamoyl-2′-deoxy-2′,2′-difluorocytidine in different solvents.

1 g of the crude 3′,5′-dicinnamoyl-2′-deoxy-2′,2′-difluorocytidine, containing 73.7% of the β-anomer and 17.5% of the α-anomer, was placed in flask and 10 ml of a solvent was added and the mixture was mixed at ambient temperature for one hour. Then, the solid was obtained by filtration, washed with 5 ml of the solvent and dried. The liquid obtained after filtering the solid and the liquid obtained after washing the solid were combined (hereinafter the mother liquor). The ratio between the β-anomer and the α-anomer in the solid and in the mother liquor was determined by HPLC and the results are summarized in Table 1.

TABLE 1

| | Content of the | Content of the α- | Content of the β- | Content of the |
|---|---|---|---|---|
| Slurrying | β-anomer in the | anomer in the | anomer in the | α-anomer in the |
| solvent | solid, % | solid, % | ML, % | ML, % |
| Acetone | 80.1 | 13.6 | 7.9 | 66.0 |
| Acetonitrile | 81.3 | 13.6 | 6.8 | 71.8 |
| Methanol:ethyl | 86.0 | 3.5 | 4.6 | 5.1 |
| acetate 1:1 | | | | |
| DCM:methanol | 93.9 | 2.8 | 15.4 | 29.8 |
| 1:1 | | | | |
| 2-propanol | 68.6 | 25.9 | 15.5 | 60.5 |
| Ethyl acetate | 81.7 | 14.1 | 4.5 | 71.7 |
| Methanol | 80.6 | 5.0 | 0.7 | 0.3 |
| DCM | 85.3 | 11.0 | 18.9 | 64.9 |

DCM = dichloromethane,
ML = the mother liquor.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing gemcitabine, comprising:
a) reacting lactol intermediate 12A with p-toluenesulfonyl chloride in the presence of a base to obtain a sulfonate intermediate of the formula 13A;
b) coupling the compound of formula 13A with N,O-bis-(trimethylsilyl)-cytosine using a catalyst in an organic solvent to obtain a mixture of α and β anomers of the 3′,5′-diprotected-N trimethylsilyl-2′-deoxy-2′,2′-difluorocytidine 17;
c) removing the trimethylsylil group and selectively precipitating the β isomer of the 3′,5′-diprotected-2′-deoxy-2′,2′-difluorocytidine; and
d) removing the protecting groups to obtain gemcitabine;

2. The process of claim 1 further comprises:
a) optionally converting the gemcitabine into a salt; and
b) optionally purifying the gemcitabine salt by crystallization.

3. A process for preparing gemcitabine or a salt thereof, the process comprising selectively precipitating the β-anomer of a 3′,5′-di-O-protected-2′-deoxy-2′,2′-difluorocytidine, removing the protecting groups to produce gemcitabine, and, optionally, converting the gemcitabine into a salt.

4. The process of claim 3, wherein the coupling reaction is carried out in an organic solvent selected from acetonitrile, ethyl acetate, n-butyl acetate, chloroform, 1,2-dichloroethane, toluene, one or more xylenes, or a mixture thereof.

5. The process of claim 4, wherein the solvent is 1,2-dichloroethane.

6. The process of claim 1, wherein the coupling reaction is carried out in the presence of a catalyst.

7. The process of claim 1, wherein the 3′,5′-protecting groups are removed by basic hydrolysis.

8. A compound of the formula 13A 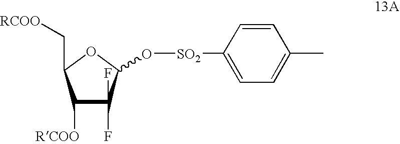
wherein R and R′ are the same or different and at least one of R and R′ is phenyl, 2-phenylethenyl (thus forming a cinnamoyl ester), 1-naphthyl, 1-naphthylmethyl, 2-methylbenzyl, 2-methylbenzyl or 4-methylbenzyl.

9. A process for separating the β-anomer from an anomeric mixture of a 3′,5′-di-O -protected-2′-deoxy-2′,2′-difluorocytidine, the process comprising:
a) dissolving a crude anomeric mixture of a 3′,5′-di-O-protected-N⁴-trimethylsilyl-2′-deoxy-2′,2′-difluorocytidine in an organic solvent and extracting with water;
b) adding an aqueous solution containing a base to the organic phase to produce a precipitate that is at least enriched in the β-anomer;
c) collecting the precipitate; and
d) optionally washing the precipitate with an organic solvent and drying.

10. The process of claim 9, wherein the 3′,5′-diprotected-2′-deoxy-2′,2′-difluorocytidine is 3 ′,5′-dicinnamoyl-2′-deoxy-2′,2′-difluorocytidine, 3′,5′-dinaphthoyl-2′-deoxy-2′,2′-difluorocytidine, 3′,5′-dinaphthylmethyl-2′-deoxy-2′,2′-difluorocytidine, 3′,5′-di-2-methylbenzyl -deoxy-2′,2′-difluorocytidine, or 3′,5′-di-4-methylbenzyl-2′-deoxy-2′,2′-difluorocytidine.

11. The process of claim 10, wherein the 3′,5′-diprotected-2′-deoxy-2′,2′-difluorocytidine is 3′,5′-dicinnamoyl-2′-deoxy-2′,2′-difluorocytidine.

12. The process of claim 9, wherein the solvent for precipitating the 3′,5′-diprotected-2′-deoxy-2′,2′-difluorocytidine is selected from dichloromethane, chloroform, ethyl acetate, 1-propyl acetate, 2-propyl acetate, butyl acetate, tert-butyl acetate, o-xylene, m-xylene, o-dichlorobenzene, toluene, and mixtures thereof.

13. The process of claim 12, wherein the solvent for precipitating the 3′,5′-di-O-protected-2′-deoxy-2′,2′-difluorocytidine is ethyl acetate.

14. The process of claim 9, further comprising removing the protecting groups of the β-anomer-enriched 3′,5′-di-O-protected-2′-deoxy-2′,2′-difluorocytidine to produce gemcitabine, and, optionally converting the gemcitabine into a salt.

15. A process for enriching the content of the β-anomer from an anomeric mixture of a 3′,5′-di-O-protected-2′-deoxy-2′,2′-difluorocytidine, the process comprising:
a) slurrying the anomeric mixture of the 3′,5′-di-O-protected-2′-deoxy-2′,2′-difluorocytidine in an organic solvent;
b) isolating the solid from the slurry;
c) optionally washing the solid with an organic solvent; and
d) optionally drying the solid.

16. The process of claim 15, wherein the 3′,5′-di-O-protected-2′-deoxy-2′,2′-difluorocytidine is 3′,5′-dicinnamoyl-2′-deoxy-2′,2′-difluorocytidine, 3′,5′-dinaphthoyl-2′-deoxy-2′,2′-difluorocytidine, 3′,5′-dinaphthylmethyl-2′-deoxy-2′,2′-difluorocytidine, 3′,5′-di-2-methylbenzyl-2′-deoxy-2′,2′-difluorocytidine, or 3′,5′-di-4-methylbenzyl-2′-deoxy-2′,2′-difluorocytidine.

17. The process of claim 16, wherein the 3′,5′-di-O-protected-2′-deoxy-2′,2′-difluorocytidine is 3′,5′-dicinnamoyl-2′-deoxy-2′,2′-difluorocytidine.

18. The process of claim 17, wherein the solvent used for slurrying the anomeric mixture of the 3′,5′-dicinnamoyl-2′-deoxy-2′,2′-difluorocytidine is dichloromethane, ethyl acetate, methanol, 2-propanol, acetone, acetonitrile, or a mixture thereof.

19. The process of claim 17, wherein the ratio between 3′,5′-dicinnamoyl-2′-deoxy-2′,2′-difluorocytidine and the solvent in the slurrying process is at least about 1:1 (g/ml).

20. The process of claim 19, wherein the ratio between 3′,5′-dicinnamoyl-2′-deoxy-2′,2′-difluorocytidine and the solvent in the slurrying process is at least about 1:10 (g/ml).

21. The process of claim 3, wherein the gemcitabine or salt thereof is obtained in a purity of at least about 98%.

22. The process of claim 21, wherein the gemcitabine or salt thereof is obtained in a purity of at least about 99.5%.

23. The process of claim 22, wherein the gemcitabine or salt thereof is obtained in a purity of at least about 99.9%.
